(19) United States
(12) Patent Application Publication
(10) Pub. No.: US 2011/0117650 A1
(43) Pub. Date: May 19, 2011

(54) Vessel and method for isolating cells from tissue portions

(76) Inventor: Neil H. Riordan, Chandler, AZ (US)

(21) Appl. No.: 12/927,276

(22) Filed: Nov. 10, 2010

Related U.S. Application Data
(60) Provisional application No. 61/280,909, filed on Nov. 10, 2009.

(51) Int. Cl. C12N 5/071; C12M 1/00

(52) U.S. Cl. 435/378; 435/283.1

(57) ABSTRACT

A vessel is adapted to be used in a procedure for isolating and collecting cells from a tissue portion using a centrifuge by providing a sealable chamber separated into two sections by a mesh screen, the chamber having a conical bottom inner surface, with a port at the apex in the lowest point in the chamber and ports at the top of the chamber, the method including placing the tissue portion in the chamber above the screen, washing the tissue, repeatedly as needed, by introducing a washing solution through a top port and draining the wash solution from the chamber through the bottom port, introducing an enzyme solution through a top port, allowing the enzyme solution to partially digest the tissue, centrifuging the vessel, and draining the isolated cells through the bottom port.

### CROSS REFERENCE TO RELATED PROVISIONAL APPLICATION

This application claims the benefit of U.S. Provisional Application No. 61/280,909, filed Nov. 10, 2009.

### BACKGROUND OF THE INVENTION

A. Field of Invention

The present invention relates generally to vessels and methods used in isolating cells from tissue portions and more particularly to a new and improved vessel and method for containing adipose tissue portions during the process of cell isolation.

B. Description of Related Art

In certain biological laboratory procedures it is desirable to isolate certain cells from gross tissue specimens. One area where this occurs is in isolation of non-adipocyte cells from adipose tissues, wherein a specimen of adipose tissue is processed to yield useful non-adipocyte cells. The current state of the art for the isolation of non-adipocyte cells from adipose tissue can be generalized as either mainly manual or fully automated. Fully automated systems have been described in and are the subjects of several patents and pending patent applications, require the purchase of the highly specialized equipment and disposable components that are quite expensive. Manual techniques can be utilized in existing laboratories, using existing laboratory equipment such as a centrifuge machine, but require large amounts of technician time, modest investments in consumable and disposable items, and carry an increased risk of contamination of the sample in order to isolate cells. Automated techniques with closed systems have a lower risk of contamination but require large monetary investments in equipment and consumable and disposable items in order to isolate cells. Conventional methods use standard conical bottom tubes that are standard laboratory disposable items and designed to withstand the forces of a centrifuge machine, a relatively inexpensive and multi-use machine that is commonly present in laboratories. The tubes are commonly used for concentration of cells and other materials by placing the material to be separated into the tube and centrifuging so materials so that the more dense materials or cells are concentrated at the tip of the tube.

For example, current manual methods of isolating non-adipose cells from adipose tissue can be described as follows:

Step one: A gross specimen of adipose tissue is transferred to the lab and portioned into multiple suitably sized portions;

Step two: each of the adipose tissue portions is placed into one of a multitude of centrifuge tubes, having a conical bottom and open or openable top;

Step three: A washing solution is added to the centrifuge tubes now containing adipose tissue and the washing solution;

Step four: The washing solution is separated from the adipose tissue by placing a multitude of tubes into a centrifuge machine, which is then operated;

Step five: The centrifuged tubes are then opened and the washing solution is decanted from the tube, leaving the adipose tissue in the tube;

Step six: Steps 3, 4 and 5 may be repeated several times as needed in order to obtain a bloodless, or near bloodless, adipose tissue portion in the tubes;

Step seven: A solution containing enzymes is then added to the tubes to digest the cleaned adipose tissue;

Step eight: The tubes containing the enzymes and adipose sample are then placed into an incubator at a temperature that is near to the optimal temperature in which the enzymes are functional;

Step nine: Once the enzymes have digested the adipose tissue, the tubes are removed from the incubator;

Step ten: The contents of the tubes are then filtered through an external filter and placed either into the original tubes or new tubes.

Step eleven: The desired non-adipose cells are separated from the filtered, digested adipose tissue by:
A. placing the tubes again into a centrifuge machine which is then operated; and
B. The tubes are then opened and non-adipose cells from the sample are then removed from the tubes by one of the two methods described as follows:
1. The lower specific gravity adipose tissue and the rest of the solution is decanted from the tube and the “pellet” of higher specific gravity non-adipocytestromal cells are aspirated from the bottom of the tube and place into another tube; or
2. The non-adipocytestromal cells are aspirated directly from the bottom of the tube.

This manual isolation procedure requires both large amounts of technician time, increasing human resource costs, and multiple openings of the tubes, increasing the risk of contamination. Therefore, what is needed, and what was invented, is an apparatus used for the isolation of certain cells from tissue portions that decreases technician time, decreases risk of contamination and provides for small monetary investments in equipment and consumable/disposable items.

It would therefore be desirable to provide a vessel that is not prohibitively expensive to manufacture and convenient to use and that will allow a method of isolation and extraction of isolated cell types from tissue portions in a procedure that is relatively expeditious and that reduces the risk of contamination during the isolation and extraction process.

### SUMMARY OF THE INVENTION

The vessel and method of the present invention is comprised of the manufacture and use of a new and improved vessel that allows for the isolation of cells from gross tissue portions.

The invented vessel comprises a self-standing conical bottom tube having two ports at or near the top of the tube and a port at the apex of the conical bottom and a built in filter screen. The self standing feature of the vessel is provided by an extension of the cylindrical wall of the tube downward past the apex of the conical bottom. The invention allows for a virtually closed system, in which, the steps of washing, digesting, and isolating the desired cells can be done through the added ports, without opening the closed tube after the gross tissue portion has been sealed therein. Specifically, the washing solution and enzyme solution can be added to the tube through the top port or ports, and the desired, isolated cells can be obtained through the port at the bottom of the tube. While the present invention was developed for use in the isolation of non-adipose cells from adipose tissue, it will be expected that the vessel and method of use can be advantageously applied to other related procedures.

The principle aim of the present invention is to provide a new and improved method and vessel for isolating cells from gross tissue portions that meets the foregoing requirements and is convenient to operate as well as economical to manufacture and use.

Other objects and advantages of the invention will become apparent from the Description of the Preferred Embodiments and the Drawings and will be in part pointed out in more detail hereinafter.

The invention consists in the features of construction, combination of elements and arrangement of parts exemplified in the construction hereinafter described and the scope of the invention will be indicated in the appended claims.

### BRIEF DESCRIPTION OF THE DRAWINGS

FIG. 1 is a perspective view of a vessel for containing tissue in accord with the present invention.

FIG. 2 is a perspective view of a disassembled vessel for containing tissue in accord with the present invention.

FIG. 3 is a cross sectional view of a vessel for containing tissue in accord with the present invention.

### DESCRIPTION OF THE PREFERRED EMBODIMENT(S)

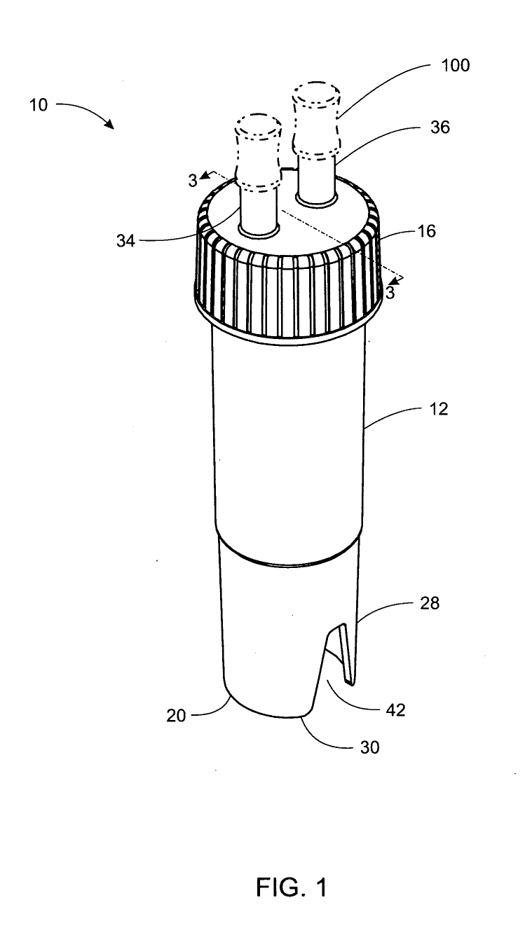

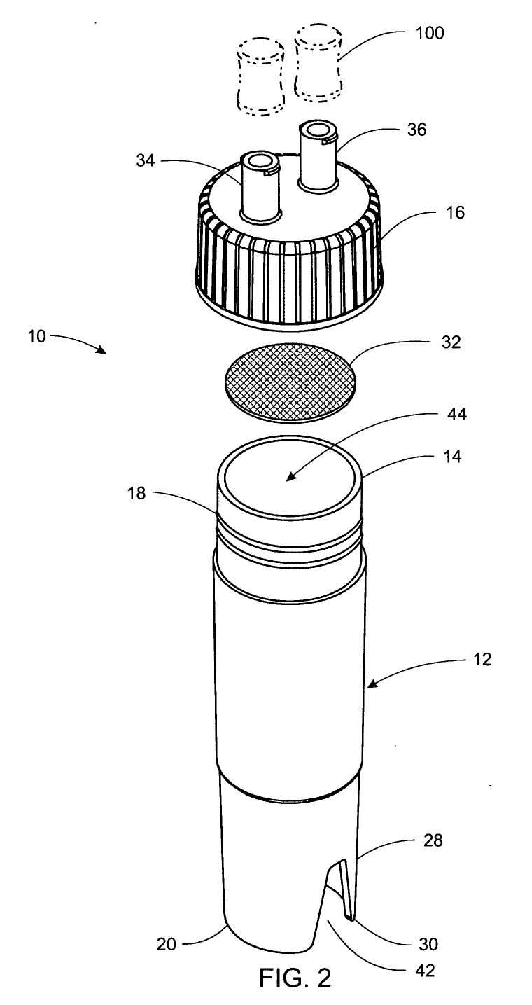

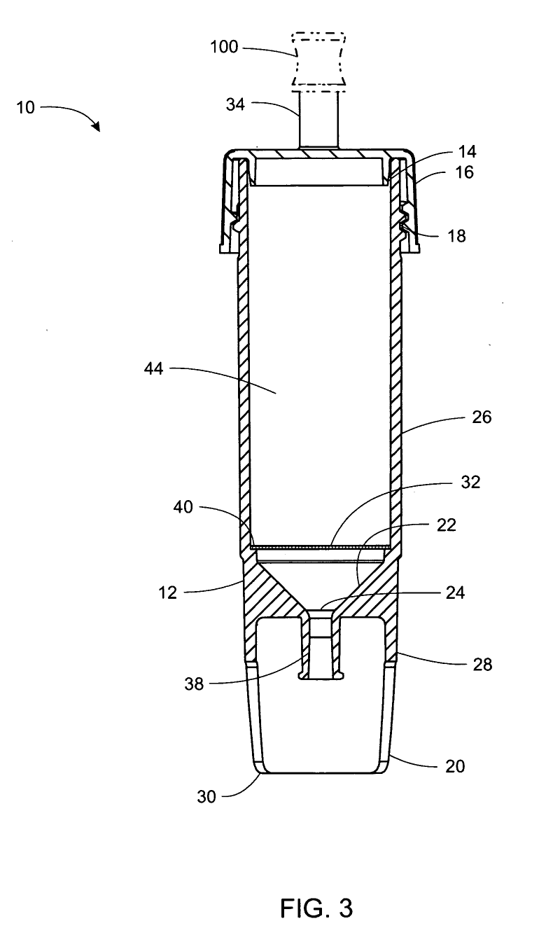

With reference to the Drawings, in which like numerals refer to like features in all figures, a vessel for containing tissue in accord with the method of the present invention. invention is generally designated by numeral 10 in FIGS. 1, 2, and 3. Vessel 10 is designed for use with a centrifuge (not shown) that comprises a plurality of sockets, sometimes referred to as buckets for receiving tubular containers of portions to be subjected to centrifugal force to stratify the contents thereof according to density. Vessel 10 comprises a generally tubular body 12 with a top end 14 and a bottom end 20. Body top end 14 is adapted to receive a cap 16 that is reopenable and resealable and may be secured by screw threads 18 or alternatively by friction fit or other acceptable means capable of preventing contamination of the contents of vessel 10. Vessel body 12 comprises a generally cylindrical outer wall 26 and, proximate to bottom end 20 a conical bottom surface 22 traverses the interior of body 12. A central chamber 44 is defined and bounded by the body wall 26, cap 16 at the top end 14 and conical bottom surface 22 at bottom end 20. Between the top 14 and the bottom surface 22, a mesh screen 32 traverses and divides interior chamber 44 of vessel 10 into an upper and a lower section. Screen 32 is formed as a circular disc and is supported by an upward facing annular shoulder 40 formed by the inside surface of the body wall 26. Screen 32 is formed of a mesh material with openings sufficiently large to allow passage of the cells to be isolated, while being small enough to prevent the tissue portion from which the cells are to be isolated and to be placed in vessel 10 from passing screen 32 under centrifugal force during the process of isolation. Screen 32 can be formed of any biologically inert material having sufficient strength to support the tissue portion above the part of the chamber 44 defined by the conical bottom surface 22 while being centrifuged.

The vessel body wall 26 extends past the conical bottom surface 22 to form a partial cylindrical skirt 28 with flat end edges 30 in a plane normal to the general longitudinal axis of vessel 10 such that the vessel 10 can be rested in an upright position on the bottom end edges 30.

Toward the top end 14 of vessel 10, two ports 34 and 36 communicate with the interior chamber 44 of vessel 10 with locking valves 100 that allow controlled entry and exit of fluid into or from vessel chamber 44. Locking valves 100 are shown in phantom for reference and in the preferred usage, self-sealing “Luer” lock ports are used to fulfill the valving function at ports 34 and 36. It will be anticipated that alternative valving systems could be used, including remote valving, if desired. In the illustrated preferred embodiment, ports 34 and 36 are formed in the top of vessel cap 16; alternatively, such ports could be formed in recesses in the outer wall 26 such that the ports and valve assemblies do not protrude past the vessel wall 11, thus allowing placement of vessel 10 in conventional centrifuge equipment. A third port 38 is located at the apex 24 of conical bottom surface 22, at the lowest point in chamber 44 and is similarly valved by acceptable means, a self-sealing “Luer” lock in the preferred embodiment.

It will be appreciated that present invention can be scaled to any size. As an exemplar only, and not as a limitation, an interior volume of that portion of chamber 44 from below cap 16 to above screen 32 of at least 500 cc is anticipated to be useful.

The present invention comprises the use of vessel 10 in a method of isolation of cells from a tissue portion. Such a method, specifically adapted for the isolation and collection of non-adipocytes from adipose tissue is described as follows:

Step one: A gross specimen of adipose tissue is transferred to the lab and portioned into multiple suitably sized portions of about one half the volume of the upper section of chamber 44 of vessel 10 from below cap 16 to above screen 32;

Step two: Each of the adipose tissue portions is placed into one of a multitude of vessels 10 through the top thereof, opening and closing cap 16 as required;

Step three: A washing solution is added to each chamber 44 through one or more of ports 34 and 36 in the top of vessel 10;

Step four: The washing solution is drained through port 38 in the bottom of the chamber 44 of vessel 10 until the adipose tissue portion is bloodless or nearly bloodless;

Step five: A solution containing enzymes formulated to at least partially digest the adipose tissue is then added to each vessel 10 through one of the top ports 34 or 36 while the bottom port 38 is closed in order to fill vessel 10 and cover the adipose tissue with the solution;

Step six: Each vessel 10 containing the enzyme solution and adipose tissue portion is then placed into an incubator at a controlled temperature that is near to the optimal temperature in which the enzymes are functional;

Step seven: Once the enzymes have digested the adipose tissue, vessel 10 is removed from the incubator placed in a centrifuge machine which is then operated to cause the cells to be isolated to collect at the apex 24 of the conical bottom surface 22.

Step eight: The non-adipose cells from the sample are then removed from vessel 10 directly from the bottom port 38.

It will be anticipated that steps three and four may be repeated as needed to obtain a clean portion of tissue. It will be further anticipated that the foregoing method may be useful if adapted to the isolation of other types of cells from tissue, without departing from the spirit of the invention.

I claim:

1. A vessel comprising a tubular body having a generally cylindrical outer wall, a closed end and an open end, and
a removable lid secured to the open end, and
a first valve secured to the vessel lid, and
a second valve secured to the closed end.

2. The vessel of claim 1, further comprising a mesh screen secured within the tubular body, traversing the interior thereof, between the closed and open ends.

3. The vessel of claim 2, wherein the closed end is shaped as a cone with a base diameter equal to the diameter of the vessel body outer wall, intersecting said wall and an apex farther from the open end than the base and the second valve is secured to the apex of said conical closed end.

4. The vessel of claim 3, wherein at least a portion of the vessel outer wall extends from the base of the conical closed end toward and past the apex of the closed end.

5. The vessel of claim 4, further comprising at least one opening in the extension of the outer wall.

6. The vessel of claim 5, further comprising a third valve secured to the vessel lid.

7. A method of isolating cells from a tissue portion comprising forming a vessel comprising a tubular body having a generally cylindrical outer wall, a closed end and an open end, a removable lid secured to the open end, a first valve secured to the vessel lid, a second valve secured to the closed end, and a mesh screen secured within the tubular body, traversing the interior thereof, between the closed and open ends, and
removing the vessel lid and placing a portion of tissue into the vessel, and
closing the vessel lid, and
adding to the vessel through the first valve a solution of enzymes capable of partially digesting the tissue, and
subjecting the vessel to force toward the closed end, and
opening the second valve to obtain cells separated from the tissue portion.

8. The method of claim 7, further comprising the step of adding a washing solution to the vessel through the first valve and draining said solution from the vessel through the second valve before adding the enzyme solution to the vessel.

9. The method of claim 8, further comprising the step of subjecting the vessel containing the tissue and enzyme solution to a temperature that is near to the optimal temperature in which the enzymes are functional for a period of time before subjecting the vessel to force.

10. The method of claim 9, further comprising the repetition of the step of adding and then draining washing solution until most of the blood is removed from the tissue before adding the enzyme solution.

11. The method of claim 10, wherein the step of subjecting the vessel and its contents to force is performed by means of a centrifuge machine.

12. A method of isolating of non-adipocyte cells from adipose tissue comprising forming a vessel comprising a tubular body having a generally cylindrical outer wall, a closed end and an open end, a removable lid secured to the open end, a first valve secured to the vessel lid, a second valve secured to the closed end, and a mesh screen secured within the tubular body, traversing the interior thereof, between the closed and open ends, and
removing the vessel lid and placing a portion of adipose tissue into the vessel, and
closing the vessel lid, and
adding to the vessel through the first valve a solution of enzymes capable of partially digesting adipose tissue, and
subjecting the vessel to force toward the closed end, and
opening the second valve to obtain cells separated from the adipose tissue portion.

13. The method of claim 12, further comprising the step of adding a washing solution to the vessel through the first valve and draining said solution from the vessel through the second valve before adding the enzyme solution to the vessel.

14. The method of claim 13, further comprising the step of subjecting the vessel containing the adipose tissue and enzyme solution to a temperature that is near to the optimal temperature in which the enzymes are functional for a period of time before subjecting the vessel to force.

15. The method of claim 14, further comprising the repetition of the step of adding and then draining washing solution until most of the blood is removed from the adipose tissue before adding the enzyme solution.

16. The method of claim 15, wherein the step of subjecting the vessel and its contents to force is performed by means of a centrifuge machine.
